(19) United States
(12) Patent Application Publication
(10) Pub. No.: US 2010/0233276 A1
(43) Pub. Date: Sep. 16, 2010

(54) COMPOSITIONS IN POWDER FORM MADE OF SOFT AGGLOMERATES OF A MICRONIZED DRUG AND OF A TWO-COMPONENTS EXCIPIENT, AND PROCESS FOR THEIR PREPARATION

(75) Inventors: Renata Raffin, Porto Alegre, Rs (BR); Paolo Colombo, Parma (IT); Fabio Sonvico, Cadorago (IT); Gaia Colombo, Ferrara (IT); Alessandra Rossi, Parma (IT); Francesca Buttini, Parma (IT)

Correspondence Address: COLLARD & ROE, P.C., 1077 NORTHERN BOULEVARD, ROSLYN, NY, 11576, US

(73) Assignee: UNIVERSITA' DEGLI STUDI DI PARMA, PARMA (IT)

(21) Appl. No.: 12/734,300

(22) PCT Filed: Oct. 26, 2007
(86) PCT No.: PCT/EP2007/061559

§ 371 Date: Apr. 23, 2010

(51) Int. Cl. A61K 9/14; A61K 9/50; A61K 9/58; A61K 9/60; A61K 9/62; A61K 9/64

(52) U.S. Cl. 424/491; 424/489; 424/490; 424/499; 424/493; 424/501; 424/497; 424/500; 424/496; 424/498; 424/502; 424/492; 424/494

(57) ABSTRACT

The described agglomeration of drug microparticles blended with excipient microparticles is a technique for the size enlargement of micronized products that could be damaged by granulation or compaction techniques. These agglomerates can be used as oral prompt or delayed-release dosage forms administered as they are or dispersed in a liquid. The composition and quantity of the excipient microparticles resulted to be the crucial factors for the agglomerate quality. Therefore, adjusting the content of surface-active agent between 8-20%, of the excipient microparticles it is possible to agglomerate microparticles of drugs that could not be agglomerated per se. Increasing the surfactant concentration in the spray-dried excipient microparticles or increasing the fraction of these excipient microparticles in the blend, the agglomeration was improved. The spray drying technique concentrates the surface-active agent on the microparticle surface. By tumbling, the surface-active agent present on microparticles excipient surface was spread to fill the inter-particle interstices of drug particles giving rise to more resistant agglomerates. This phenomenon occurred also by vibration; the production in this case was quicker.

### FIELD OF INVENTION

The present invention refers to a solid pharmaceutical product in form of soft agglomerates containing drug microparticles administered for prompt or delayed-release. Drug microparticles are unsuitable to administer because they are too small for flowing and packing properly, making difficult the pharmaceutical dosage form manufacturing. Soft agglomerates are clusters of primary microparticles in which the links between particles are easy reversed by the contact with water or by air turbulence. Soft agglomerates are useful as dosage forms since they flow and pack easily. A novel technique has been found to prepare soft agglomerates without affecting the stability and the structure of the drug primary microparticles. Drug microparticles, non-agglomerating per se, were preliminary blended with spray-dried microparticles made of a mixture of a support substance and a surface-active agent; the powder blend was agglomerated by tumbling or sieve vibration. It has been found that the amount of surface-active agent in the excipient microparticles and the ratio between drug and excipient microparticles determines the agglomeration. Moreover the surface-active agent located in spray-dried microparticles at the particle surface, creates the most favorable conditions for particle binding in the agglomerate.

### TECHNICAL BACKGROUND

Proteins or peptide have a relevant position in drug therapy, but the difficulty to prepare stable dosage forms with these delicate drugs reduces the possibility of their administration avoiding injection. Polymeric drug microparticles cover a relevant position among drug delivery systems, considered that they are employed to control drug release, to modify drug uptake or to improve biological availability of drug. However, the attainment of these biopharmaceutical characteristics requires pharmaceutical preparations that facilitate the administration of the drug dose. These aspects are particularly stringent when a solid dosage form has to be prepared. In solid dosage form manufacturing, particles might be fine for drug delivery, but coarse enough for facilitating solid dosage form preparation. One solution to this constrain is size enlargement but, often, the transformation of drug microparticles in large solid structures involves steps of granulation and compaction, leading to irreversible modifications of the drug microparticle size or its physico-chemical characteristics. In some cases drug microparticles are coated by a polymeric film for giving to preparation the capability to drive the performance of the micro-structure, in particular the drug delivery. For example, numerous drug particles are coated with a membrane in order to control the release rate or to delay their delivery in a precise site, like the small or large intestine. This is the typical case of gastro-resistant preparations in which the drug must be protected from gastric environment during the passage of dosage form in the stomach. The classical solution in this case is the manufacturing of tablets or pellets coated by a membrane insoluble in acid environment, but the manufacturing procedures can affect the microparticle delayed-release characteristics. The only possibility in the cases in which the coated microparticles suffer for manufacturing procedures, is to administer the gastro-resistant microparticles directly as powder or dispersed in a non-solvent liquid. Similarly, the advent of advanced therapy made available numerous biotechnological substances to be administered in solid forms. Since these bio-substances suffer during the size enlargement procedures for mechanical stresses, humidity or heat, pharmaceutical products able to maintain the stability and performance have to be prepared. Furthermore, there are many patients that are unable to swallow solid dosage forms such as children or old people. For them microparticulate drug preparations would be the easy solution for facilitating dose intake.

Therefore, the direct administration of microparticles is the only possibility for several drug administration requirements. Unfortunately, the dosage form manufacturing is rendered difficult by the small size of particles that leads to powders with high bulk volume and problematic flow for dosage form manufacturing. The metering during capsule filling or sachet dosing or other techniques involving the flow and packing properties of powder are strongly impeded by the properties of microsize powders. It is highly desirable to find a powder that is fine and large at the same time in order to couple the benefits of the large size for manufacturing and of the small size for drug delivery.

It has been said that this technological size problem could be tackled by soft agglomeration, a process in which the powder size is enlarged by constructing weak clusters of primary microparticles (Russo P., Buttini F., Sonvico F., Bettini R., Massimo G., Sacchetti C., Colombo P., Santi P.—Chimeral agglomerates of microparticles for administration of caffeine nasal powders—J. Drug Deliv. Sci. Tech., 14, 449-454, 2004; Russo P., Sacchetti C., Pasquali I., Bettini R., Massimo G., Colombo P., Rossi A.—Primary microparticles and agglomerates of morphine for nasal insufflation—J. Pharm. Sci., 95, 2553-2561, 2006).

In fact, soft agglomerates, as consistent ensemble or cluster of small particles, are free flowing and predictable in packing. They are easily broken down by air turbulence or water uptake, reconstituting the original size of microparticles. This concept has already been applied in inhalation product manufacturing and it was based on the electrostatic attraction between particles. Drug microparticles have been also described that in dependence on their composition can be directly assembled in agglomerates by tumbling (Russo P., Buttini F., Sonvico F., Bettini R., Massimo G., Sacchetti C., Colombo P., Santi P.—Chimeral agglomerates of microparticles for administration of caffeine nasal powders—J. Drug Deliv. Sci. Tech., 14, 449-454, 2004).

These agglomerates were weak enough to reconstitute the primary particle size, but resistant enough to be transported and processed during powder manipulation, allowing accurate metering in dosing devices. Weak cohesion bonds due to capillary, Van der Waals or electrostatic forces hold together the primary particles in soft structures. The quantity and nature of these interactions, as well as the method of production, determine the agglomerate formation. Thus, the availability of powder agglomerates would open the possibility to perform alternative strategies of drug administration without affecting the properties of the drug microparticles, in particular their physico-chemical structure or coating integrity. Unfortunately, not always the agglomeration of drug microparticles is a feasible process because several microparticle powders are not agglomerating per se.

For example, many drugs are orally administered in enteric dosage forms, due to instability in acidic environment or for targeting distal parts of the intestine (colon). The dosage forms of these drugs are in general coated tablets or coated hard pellets manufactured by means of complex processes. In addition, these forms are less useful for administration to patients having difficult to swallow the dosage form. Drug-loaded microparticles prepared by spray drying with enteric polymers have been described for allowing the gastro-protection of drug in acid environment. These gastro-resistant microparticles kept the stability of encapsulated drug in acid. However, the technological properties of the powder (flow and packing) were very poor not allowing a precise and accurate dose metering. Granulation and compaction are classically used for making larger free flowing particles, but these processes could damage the enteric coating microparticles, thus exposing the drug to acid environment. A solution available for the expert man in order to circumvent these obstacles remains the preparation of soft agglomerates of drug microparticles. This is considered a suitable strategy for maintaining the primary microparticle integrity in dosage form. Unfortunately, the gastro-resistant microparticles are not able to agglomerate.

Also several biotechnological substances would be more accepted if given by routes alternative to injection administration. Among these substances insulin is a drug considered in numerous studies for inhalation or oral administration in solid dosage forms. Microparticles of insulin were prepared by various techniques because they dissolve quickly, but the obtained powders require dosage forms manufactured in consideration of the low dose, the small size of particles and the poor stability of the substance. For these substances the pharmaceutical product fabrication must avoid the operations involving heating, wetting or compacting since they affect drug stability.

All these drug therapy aspects have an additional problem when the administration to special population is requested. In this case a dosage form easy to swallow is demanded that is a solid dosage form to be given in water dispersion or directly dissolved in mouth. In fact, it is not easy to prepare a dosage form containing unstable or coated drug microparticles, able to be dispersed in water in order to give rise to a smooth suspension that could be swallowed without gritting feeling by patients and maintaining the stability.

Agglomerates of spray-dried primary microparticles of morphine obtained by tumbling have been described. Due to morphine spray-dried stability, a mixture of morphine crystals with spray-dried excipient microparticles was agglomerated. The easiness of agglomeration of this mixture was due to the peculiar shape of morphine acicular crystals since the flat crystal faces determine many contact points between particles. The regular shape, the typical crystal electrostatic properties and hygroscopicity created enough weak bonds between morphine crystals to provide the agglomerate formation.

Primary particles of insulin have been prepared for inhalation. These microparticles have been used in inhalation product (Exubera, Pfizer). The primary microparticles of insulin could be given by oral or nasal administration but this requires a formulation in solid dosage form that can affect the stability of the drug. Agglomerates of primary particles of insulin could have been the solution of this problem but the insulin primary microparticles do not agglomerate.

Thus, coated or biotech microparticles are different from crystals since they are amorphous, irregular or spherical in shape and non-agglomerating per se due to weak particle-particle attraction. Unfortunately, not all the microparticle powders have the properties of crystals and their agglomeration is not feasible by direct tumbling of powder. We have now found, and it is the object of the present application, a new technique and new materials that can be applied for soft agglomeration i.e., size enlargement or mass increase, of coated drug microparticles or of delicate microparticles of drugs, in view of direct administration or water dispersion of produced powders. We have found that the employment of a new cohesive excipient microparticle powder mixed with the drug primary microparticles introduces enough cohesion between particles to provide a resistant agglomerated structure.

### SUMMARY OF THE INVENTION

In the present patent a new technology for manufacturing solid drug dosage forms in which the drug microparticles, having a peculiar biopharmaceutical function, are agglomerated in soft structures is described. This is particularly beneficial for those drug microparticles that cannot be transformed or processed in solid dosage forms without affecting in irreparable way the particle biopharmaceutical characteristics. The agglomeration of microparticles that do not have enough intrinsic cohesion for creating an agglomerated solid structure is an important problem. The innovative solution for these drug microparticles does essentially consist in their embedding in agglomerated solid dosage form, in which the poor cohesive properties of drug microparticles, not agglomerating per se, have been improved by mixing them with spray-dried cohesive excipient microparticles. This agglomeration technique did not affect particle integrity and drug delivery.

One essential aspect of the invention resides in the finding of a new excipient microparticle structure obtained when two components, one having a support role for structuring the agglomerate and the other one having a surface-active role for facilitating the dissolution and drug release from the agglomerate, were spray-dried in particular conditions of solution and dispersion. The finding was the unexpected positioning in the excipient microparticle structure of the surface-active component. We unexpectedly found that the agglomeration of poorly cohesive drug microparticles was determined by the location of surface-active agent at the external surface of excipient microparticles. This made the excipient microparticle composition and their ratio to drug microparticles in the blend the crucial element for agglomerate formation according to the invention.

Therefore, the invention resides in the composition and agglomerate formation mechanism of excipient microparticles that when blended with non-agglomerating drug microparticles, introduces into the blend enough cohesion to allow all particles to adhere each other with formation of free flowing and resistant solid agglomerates. Their structure, however, remains sufficiently weak to restore the original size of the composing microparticles after the intervention of water and, at the same time, sufficiently resistant to face the manipulation of the drug dosage metering. The water dispersion of these soft agglomerates gives rise to a very smooth active drug suspension, suitable for administration to special patients.

As said, we unexpectedly found that the excipient microparticle composition is determinant for the agglomeration and successive microparticle water dispersion, due to the peculiar positioning of the surface-active substance component on the external surface of the excipient microparticles. This positioning gives to the surfactant, generally employed for accelerating dissolution, the new role of binding agent. The discovery of the binding role of this class of excipients in the agglomeration process allows to the excipient microparticles to mend the drug microparticles cohesion and adhesion defects. In addition, the presence of the surface-active adjuvant allows the easy dispersion of agglomerate in water, transforming the solid preparation in a liquid suspension easy to administer to patients having difficulty to swallow solid medicines.

Furthermore, thanks to the cohesion introduced by surface-active component, a new mechanical process for agglomerating powder was made possible. In fact, it was possible to obtain agglomerates by vibrating the powder blend of a sieve stack; in particular, the sieve vibration process made faster the preparation of agglomerates in large quantity and implemented the classical tumbling procedure.

In summary, new adhesive excipient microparticle powders have been made using two main components: a support substance such as sugar, sugar derivative or polymeric carbohydrate, and a surface-active substance having liquid or semisolid consistency. A solution or dispersion of these two components in appropriate ratio was spray-dried in order to prepare micronized particles made of the mixture of the two components. Surprisingly, these primary microparticles exhibit cohesive properties that can be used for improving the poor cohesion of other particle populations.

We further found that the performance of excipient microparticles in promoting the cohesion of other microparticle population depended on the percent of the surface-active agent and on the ratio of the excipient microparticle in the blend. It was also found that manufacturing the excipient microparticles by spray drying, the surface-active component was not uniformly distributed in the microparticle structure, but remained mainly concentrated on the surface of microparticles. This location is essential for the object of the present invention and even more relevant in dependence on the percentage of surface-active agent in the excipient microparticle composition. Using the analytical techniques of atomic force microscopy and scanning electron microscopy analysis, it was discovered that the surface of excipient microparticles is coated with a layer of the surface-active agent. This was surprising since the surface-active ingredient was expected to be uniformly distributed in the structure or body of microparticle. The thickness of this layer was proportional to the content of surface-active agent in the spray-dried mixture. In addition, we found that during the mechanical processes for agglomerate manufacturing, the surface-active substance located at the microparticle surface migrates in consequence of the stresses introduced by vibration or by tumbling. This migration displaces and accumulates the surface-active component in between particle interstices improving the inter-particle cohesion, and consequently reinforcing the internal structure of agglomerates. This is clearly shown in FIG. 1 where a picture of an agglomerated particle is reproduced.

### DESCRIPTION OF THE FIGURES

The invention described and claimed in the present patent application is further illustrated, without limiting the same, in the enclosed Figures, which refer to some non-exhaustive examples of the possible embodiments of the invention and in which:

FIG. 1 shows SEM images of agglomerate of example 1 prepared by vibration (a,b) and tumbling (c,d) at magnifications of 100×(a,c) and 1,000×(b,d).

FIG. 2 shows the atomic force microscopy images of the surface of spray dried microparticles composed of mannitol/lecithin 85:15 prepared according to the spray drying technique of this patent.

FIG. 3 shows (A) one agglomerate and (B) an enlargement of insulin microparticles mixed with excipient microparticles prepared accordingly to example 2

### DETAILED DESCRIPTION OF THE INVENTION

With the aim of overcoming the above-identified drawbacks, of fine particles, the inventors of the present application have found a way to obtain soft agglomerates from non-agglomerating powders employable as dosage forms for releasing microsize particles, whose handling on industrial scale is markedly improved.

The invention consists in a new dosage form for drug administration useful for various routes such as nasal, oral, buccal or dermal. The new dosage form is made of soft agglomerates of microparticles obtained by tumbling, but in preferred way by vibrating a blend of drug microparticles mixed with peculiar excipient microparticles. The reason for agglomerating drug microparticles is principally due to the need to preserve the properties of microparticles without affecting their physico-chemical characteristics, avoiding processes detrimental for the drug characteristics. This is in particular evident with drug particles coated for controlling or delaying drug release or with biotechnological substances that suffer for humidity and high temperature.

Drug microparticles, prepared by the procedures capable to reduce the size of the particles are not always capable to agglomerate. The goal of avoiding the damaging of the drug microparticles in condition of industrial fabrication has been in this patent attained through the provision of an improved technology consisting in mixing the drug microparticles with original excipient microparticles,

As a consequence, the invention describes the manufacturing of new agglomerates of drug microparticulate powders by mixture with a special microsize powder, defined excipient microparticulate powder, obtained by spray drying a solution or dispersion of adjuvant substances. In details, the microparticulate drug powder was blended with a percentage of the excipient microparticulate powder in an appropriate mixer for powders. The obtained blend is transferred in a rotating drum or preferentially on a vibrating sieve stack and rotated or vibrated for a fixed period of time. The mechanical tumbling or vibration favors the cohesion between drug and excipient microparticles and gives rise to a collection of agglomerates composed of stuck microparticles, having size two orders of magnitude higher than primary microparticles. Agglomerates are collected and size calibrated by sieving generally soft aggregates of a diameter between 80 and 1500 μm, preferably between 100 and 1000 μm, are separated. The formation of agglomerates and yield of the process is dependent on the composition and the percentage of excipient microparticles in the blend.

Therefore, the core of the invention is the structure presented by the microparticles of excipient that in turn depends on the composition. The excipient microparticulate powder was prepared by spray drying a water/alcohol solution/dispersion of at least two types of adjuvant. The first adjuvant gives the structure to the spray-dried microparticle, in order to allow the formation of the agglomerate mass after mixing with drug microparticles. It is defined the “support” component. The second adjuvant is a surface-active agent used for favoring the dissolution and release from the agglomerate of the microparticle drug. In particular, it has been demonstrated that when the surface-active adjuvant is present in the composition above a certain amount, during spray drying it deposits mainly on the surface of excipient microparticles. This position determines the interstices between the particles in the agglomerate to be filled by the adjuvant that, since it is semi-solid, migrates under the effect of tumbling or vibration movements. This creates the cohesion between microparticles of drug and excipient without affecting the release of drug, despite the original role of surface-active agent in the agglomerate was to facilitate the release of microparticles.

In the excipient microparticle composition the support substance can be present in an amount from 70 to 99% and the surface-active agent in a percentage from 1 to 30%. In the most preferred composition the surface-active agent is present in an amount from 10 to 20%.

Concerning the support component used in the excipient as the adjuvant for the construction of the solid microparticles and agglomerates, useful substances are:
sugars like glucose, lactose, sucrose, trehalose, maltose, mannose or fructose;
polyalcohols like mannitol, xylitol, sorbitol, lactitol;
amino-sugars like glucosamine;
polysaccharides like starch, dextranes, dextrines, cyclodextrines and derivatives, maltodextrines;
polymers like cellulose and its derivatives, chitosan, alginic acid ant its salts, pectine, starch, guar gum, xantan gum, carrageenan, polyethylene oxide, polymethacrylates;
peptides and proteins as albumin, gelatin;
mixtures of the above mentioned excipients

Concerning the surface-active component, useful substances are:
phospholipids and their mixtures, like lecithins;
fatty acids and their salts, esters and corresponding alcohols, such as aluminum stearate, sodium stearyl fumarate stearic, lauric, palmitic, linoleic acid, myristic acid, cetostearyl alchohol, glyceryl monostearate, glyceryl palmitostearate, polyoxyethylene stearates, sucrose palmitate (Sucrodet);
ionic and non-ionic surfactants, such as poloxamers (Pluronic), sorbitan fatty acid esters (Span), polyoxyethylene sorbitan fatty acid esters (Tween);
polymers, such as polyethylenglycols with molecular weight higher than 1500, polyoxyethylene alkyl ethers and polyoxyethylene castor oil derivatives (Cremophor), polyoxiethylenated glycerides;
mixtures of the above mentioned excipients.

The preparation of excipient microparticulate powder is performed, in a typical but not exclusive embodiment of the invention, by spray drying a dispersion obtained by mixing a water solution of the support substance with an alcoholic solution of the surface-active agent. The dilution of the alcoholic solution in water determines the formation of a colloidal dispersion of the surface-active agent in the prevailing water solution. The spray drying process of this dispersion produces microparticles that resulted coated by the surface-active adjuvant. This result was not predictable since the formation of a homogeneous particle in term of composition was expected.

In a typical but not exclusive composition, the excipient microparticulate powders were prepared by spray-drying different solutions of mannitol and lecithin. The lecithin contents of the spray-dried powders obtained were in the range 10-15%. Spray-dried excipient microparticles were prepared accordingly to the following procedure: mannitol was dissolved in 90 mL of water; lecithin was dissolved in 10 mL of ethanol at 40° C. and mixed with mannitol solution giving an opalescent mixture. Mannitol and lecithin ratios used were 90:10, 87.5:12.5 and 85:15 (w/w) and the solid concentration was 4% (w/v). All the solutions were spray-dried using a Buchi Mini Spray Dryer B-191 in the following conditions: inlet temperature 90° C., outlet temperature 38-40° C., feed rate 6.0 mL/min, nozzle diameter 0.7 mm, drying air flow 600 L/h.

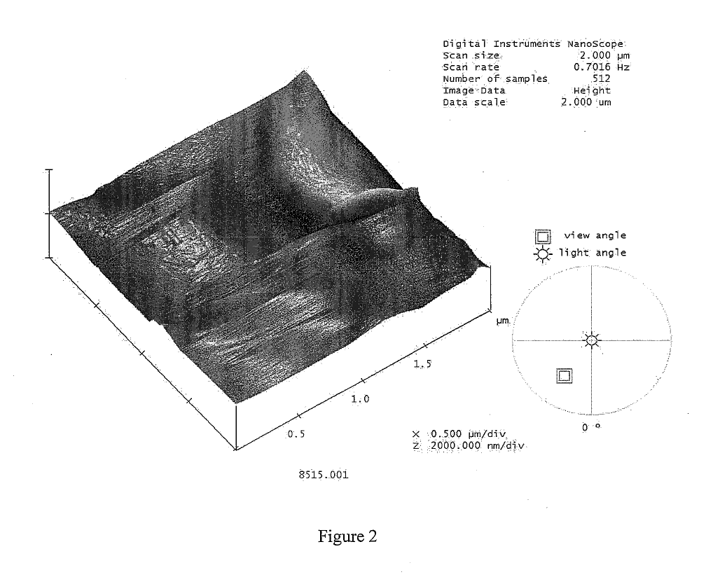

The median volume diameters of the three powders were 3.6, 3.7 and 3.7 μm respectively. These spray-dried powders presented high bulk volumes (bulk density around 0.2 g/cm³) typical of fine powders, poor packing and did not flow. The excipient powders prepared showed typical spray-dried round particles and evidenced a tendency to form clumps as the content of lecithin increased. In the microparticles, lecithin was located at the surface. Its presence on particle surface was discovered analyzing the excipient microparticles containing the lecithin by means of atomic force microscopy and x-ray microanalysis. In comparison with particles made of mannitol alone, the surface of the particle containing lecithin appeared coated with a curly layer of lecithin. This was confirmed by x-ray microanalysis searching phosphorus on the surface of the microparticles mannitol/lecithin. The results obtained revealed the presence of phosphorus (P) peak among the components of the particle surface in comparison with the mannitol alone particles where there was no P peak. This peak was more intense in case of microparticles with the highest lecithin content. Thus, lecithin accumulated on the microparticle surface, in particular in those particles having the highest content. The picture of this unexpected particle is presented in FIG. 2.

A further object of the present invention is a process for obtaining the herein described agglomerates comprising the mixing of drug microparticle with the excipient microparticles and treating the obtained blend in a stack of appropriate sieves in order to vibrate the blend favoring the cohesion between the drug and excipient microparticles. It was discovered that this procedure was faster than the classic tumbling process and the quality of the agglomerates was possible only with the excipient microparticulate powder described in this patent.

### Examples

### Example 1

In order to illustrate the invention, in this example the preparation of an agglomerated powder for oral administration of a drug that requires to be protected from the gastric environment was described. This description is not limitative to this drug microparticulate powder since the procedure can be applied to all the situations in which the drug particles must be protected from the dosage form fabrication processes. The gastro-resistant microparticles need to be maintained in their integrity during dosage from manufacturing. Granulation and compaction are considered options for manufacturing the dosage form, since drug-loaded microparticles could be damaged. Soft agglomeration was applied to improve the poor packing and flow of drug microparticle powders. The objective was to maintain the powdered size and the intestinal release properties in the final dosage form.

The example describes the preparation of agglomerates made of pantoprazole gastro-resistant microparticles. Pantoprazole, a drug for the treatment of gastric ulcers, was prepared as gastro-resistant microparticles by spray drying a solution of the drug and Eudragit S100 as described in Raffin R. P., Guterres S. S., Pohlmann A. R., Re M. I.—Powder characteristics of pantoprazole delivery systems produced in different spray-dryer scales—Drying Tech., 24, 339-348, 2006.

Microparticles had mean diameter of 15.6 μm and contained 20% (w/w) of pantoprazole. The powder bulk volume was high (bulk density 0.25 g/cm³) and the flow was very poor.

These pantoprazole microparticles could not be directly agglomerated; then, blends of pantoprazole gastro-resistant microparticulate powder with mannitol/lecithin spray-dried powders containing 10.0, 12.5 and 15.0% (w/w) of lecithin respectively, were prepared in order to manufacture soft agglomerates. Soft agglomerate powders were obtained from blends between pantoprazole and excipient microparticles. The agglomerates prepared using tumbling or vibration of 1:1 ratio microparticle mixtures and containing the lowest amount of lecithin, presented quite low agglomeration yields. In addition, pantoprazole microparticles were poorly incorporated into these agglomerates. The agglomerates prepared at ratio 1:1 using the excipient microparticles containing more lecithin (12.5% w/w) showed an agglomeration yield of 64%, but the drug loading was still incomplete. Furthermore, the agglomerates, prepared with 1:1 ratio of pantoprazole microparticles and excipient microparticles with a lecithin concentration of 15.0% w/w, showed high agglomeration yields and drug loading was fairly complete. The agglomerates, in which the ratio between the two populations of microparticles was 1:2, gave agglomeration yields between 61.5 and 84.0% and the pantoprazole microparticles were completely embedded into agglomerates. Summarizing, as the amount of lecithin in the blend increased, due to either the lecithin content in the excipient microparticles or to the increased ratio of excipient microparticles, the agglomeration yield and pantoprazole loading improved. Moreover, the process was faster performing the agglomeration by vibration.

For soft agglomerate preparation, different mixtures of drug microparticles and excipient microparticles were prepared in Turbula apparatus. Each mixture was split into two portions and agglomeration was performed by two techniques.

In case of tumbling process, five grams of the mixture of pantoprazole and excipient microparticles were rolled into a Bakelite cylindrical jar (diameter 5.0 cm, length 4.4 cm), rotating at 45 rpm on the cylinder axis tilted at 90°. At intervals of 30 min, the agglomerates between 106 and 850 μm were collected by sieving. The entire process lasted 3 h.

In case of vibration process, five grams of the mixture of drug microparticle and excipient microparticles were put on the top of a stack of two sieves with nominal apertures of 850 and 106 μm respectively (10 cm diameter sieves), which was vibrated for 5 minutes on a laboratory sieve shaker. Agglomerates between 106 and 850 μm were collected. The non-agglomerated powder were reprocessed 5 times, crushing the larger agglomerates each time. The entire process lasted less than 1 h.

The agglomerates showed bulk densities around 0.30 g/cm³, corresponding to a loose packing arrangement of particles. The tapped density values of agglomerates were close to bulk values and the compressibility indexes were around 16. Agglomerate powder beds are very porous, with values ranging between 76% and 82%, a condition that favors fast water penetration. Thus, the agglomeration process, determining the organization of two different populations of particles in the globular structure, favored the packed arrangement of powder bed over primary microparticle powders. The agglomerates could be classified as free-flowing powders. In summary, the agglomerates showed characteristics of packing arrangement and flowing ability very favorable for handling and metering the microparticles.

Pantoprazole soft agglomerates had a very low resistance to crushing and the tensile strength values were between 30 and 52 mN/mm². Tumbling produced more resistant agglomerates, but the production rate was slower than with vibration procedure. In summary, the agglomerates prepared presented good resistance during flowing and poor resistance when compressed. Based on these features, they are suitable for filling hard gelatin capsules in view of oral administration.

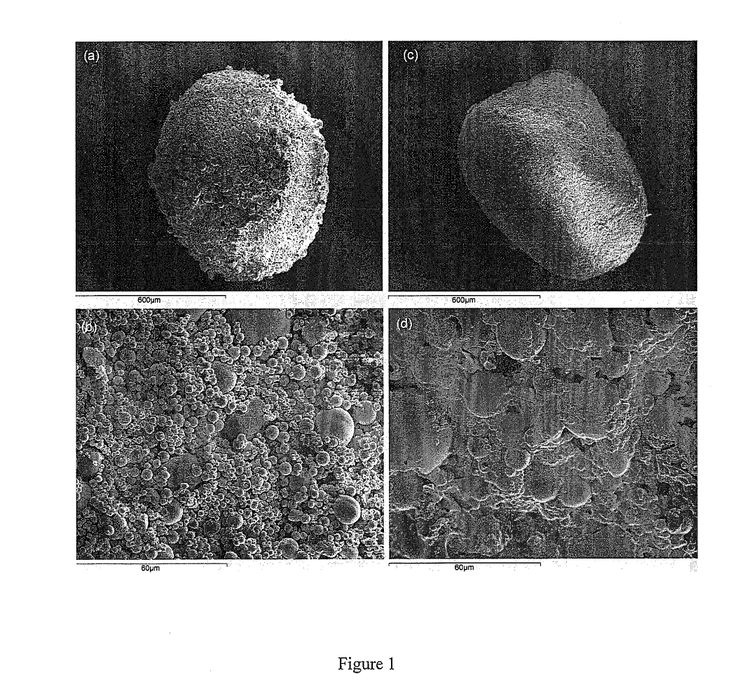

In order to elucidate the agglomerate structure, SEM analysis was performed. Photomicrographs of surface of agglomerate prepared by vibration (FIG. 1a,b) evidenced that they consist of an assembly of small (excipient) and larger (pantoprazole) microparticles. The surface of agglomerates prepared by tumbling was smoother and the inter-particle space was filled of lecithin present in excipient microparticles. This created solid bridges between the particles. This was particularly evident for the agglomerates containing the excipient microparticles with higher content of lecithin (FIG. 1c,d).

### Example 2

The preparation of insulin microparticle agglomerates to be used for oral, buccal or nasal delivery of insulin are here described. Insulin solutions to be spray dried were prepared dissolving 1 g of insulin in CH₃COOH 0.4M. The pH 3.3 was chosen after experimental observation that higher values determined the precipitation of the hormone. The concentration of the total solids in the solution was kept at 1% w/v (eg. 1 gr in 100 ml).

Insulin spray dried powders were prepared employing a Mini Spray Drier Büchi 190. Briefly, an inlet temperature of 120° C., a drying air flow rate of 600 l/h, a solution feed rate of 3.25 ml/min and an atomizing air pressure of 6 bar were selected. Dried microparticles were collected via a high efficiency cyclone. These insulin microparticles have a corrugated aspect and could not be directly agglomerated; then, blends of mannitol/lecithin 85:15 spray-dried powders with insulin microparticulate powder were prepared in order to manufacture soft agglomerates using vibration. Soft agglomerate powders were easy obtained from blends between insulin and excipient microparticles in 1:9 ratio. The agglomerates prepared showed high yield and insulin loading was complete. Mixtures of insulin microparticles and excipient microparticles were prepared in Turbula mixer. Five grams of the mixture of drug microparticles and excipient microparticles were put on the top of a stack of two sieves with nominal apertures of 850 and 106 μm respectively (10 cm diameter sieves), which was vibrated for 5 minutes on a laboratory sieve shaker. Agglomerates between 106 and 850 μm were collected. The process was done 5 times reprocessing the non-agglomerated powder and crushing the larger agglomerates. The entire process lasted less than 1 h.

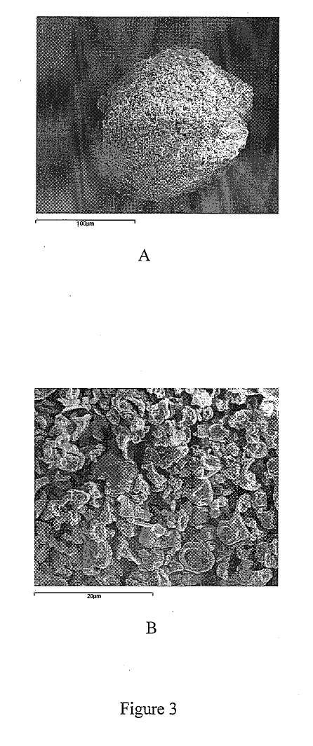

The properties of the agglomerates obtained are similar to the ones of the agglomerates prepared in previous example. The picture of the agglomerate surface shows the insulin corrugated microparticles embedded in the excipient microparticles (FIG. 3).

### REFERENCES

[1] Russo P., Buttini F., Sonvico F., Bettini R., Massimo G., Sacchetti C., Colombo P., Santi P.—Chimeral agglomerates of microparticles for administration of caffeine nasal powders—J. Drug Deliv. Sci. Tech., 14, 449-454, 2004.

[2] Russo P., Sacchetti C., Pasquali I., Bettini R., Massimo G., Colombo P., Rossi A.—Primary microparticles and agglomerates of morphine for nasal insufflation—J. Pharm. Sci., 95, 2553-2561, 2006.

[3] Adi H., Larson I., Chiou H., Young P., Traini D., Stewart P.—Agglomerate strength and dispersion of salmeterol xinafoate from powder mixtures for inhalation—Pharm. Res., 23, 2556-2565, 2006.

[4] Boerefijn R., Ning Z., Ghadiri M.—Disintegration of weak lactose agglomerates for inhalation applications—Int. J. Pharm., 172, 199-209. 1998.

[5] Tsantilis S., Pratsinis S. E.—Soft- and hard-agglomerate aerosols made at high temperatures—Langmuir, 20, 5933-5939, 2004.

use in the management of acid-related disorders—Drugs, 63, 101-132, 2003.

[6] Raffin R. P., Guterres S. S., Pohlmann A. R., Re M. I.—Powder characteristics of pantoprazole delivery systems produced in different spray-dryer scales—Drying Tech., 24, 339-348, 2006.

1-9. (canceled)

10. Medicinal preparation in form of powder of soft agglomerates having particle size of 106 to 850 mm, for oral, buccal or nasal administration, comprising a blend of micronized, microencapsulated or other micron-sized multiparticulated drug mixed with adhesive excipient microparticles.

11. Medicinal preparation of claim 10, wherein in said blend the ratio between the multiparticulated drug and the adhesive excipient microparticles is of 0.1:99.9 to 75:25.

12. Medicinal preparation of claim 10, wherein said soft agglomerates are prepared by tumbling the blend of drug and excipient microparticles in a suitable container for a period of time ranging from few seconds to 3 hours and afterwards collecting the agglomerates of the desired particle size by sieving.

13. Medicinal preparation of claim 10, wherein the adhesive excipient microparticles comprise a support substance in an amount of 70 to 99% by weight and a surface-active agent in an amount of 1 to 30% by weight.

14. Medicinal preparation of claim 13, wherein the surface-active agent is in an amount of 10 to 20%.

15. Medicinal preparation of claim 13, wherein the support substance is chosen from sugars, polyalcohols, amino-sugars, polysaccharides, cellulose and its derivatives, chitosan, alginic acid and its salts, pectine, starch, guar gum, xantan gum, carrageenan, polyethylene oxide, polymethacrylates, peptides and proteins, and mixtures thereof.

16. Medicinal preparation of claim 15, wherein the support substance is selected from the group consisting of glucose, lactose, sucrose, trehalose, maltose, mannose or fructose, mannitol, xylitol, sorbitol, lactitol, glucosamine, starch, dextrates, dextrines, cyclodextrines and derivatives, matodextrines, cellulose and its derivatives, chitosan, alginic acid and its salts, pectine, starch, guar gum, xantan gum, carrageenan, polyethylene oxide, polymethacrylates, albumin, gelatin, and mixtures thereof.

17. Medicinal preparation of claim 13, wherein the surface-active substance is chosen from phospholipids, lecithin, fatty acids and their salts, ester and corresponding alcohols, ionic and non-ionic surfactants, polyethylene glycols, polyoxyethylene alkyl ethers, polyoxyethylene castor oil derivatives, polyoxiethylenated glycerides, and mixtures thereof.

18. Medicinal preparation of claim 17, wherein the support substance is selected from the group consisting of aluminum stearate, sodium stearyl fumarate stearic, lauric, palmitic, linoleic acid myristic acid, cetostearyl alchohol, glyceril monostearate, glyceriyl palmitostearate, polyoxyethylene steararters, sucrose palmitate, poloxamers, sorbitan fatty acid esters, polyoxyethylene sorbitan fatty acid esters, polyethylene glycols, polyoxyethylene alkyl ethers, polyoxyethylene castor oil derivatives, polyoxiethylenated glycerides, and mixtures thereof.

19. Medicinal preparation of claim 13, wherein the adhesive excipient microparticles consist of a support substance in an amount of 70 to 96% by weight and a surface-active agent in an amount of 4 to 30% by weight.

20. Medicinal preparation of claim 19, wherein the adhesive excipient microparticles consist of a support substance in an amount of 80 to 92% by weight and a surface-active agent in an amount of 8 to 20% by weight.

21. Medicinal preparation of claim 10, wherein the surface-active agent in the adhesive excipient microparticles is concentrated at the external surface of said microparticles.

22. Medicinal preparation of claim 10, wherein the excipient microparticles are obtained by the steps of dissolving the support substance in 90 mL of water and surface-active substance in 10 mL of ethanol at 40° C., mixing the two solutions thus giving an opalescent mixture, and spray-drying the latter at the solid concentration of 4% (w/v).
